(19) United States
(12) Patent Application Publication
(10) Pub. No.: US 2018/0196130 A1
(43) Pub. Date: Jul. 12, 2018

(54) ULTRASOUND PROBE UNIT AND ULTRASOUND DIAGNOSTIC APPARATUS

(71) Applicant: Konica Minolta, Inc., Tokyo (JP)

(72) Inventor: Eiichi Okawa, Kanagawa (JP)

(73) Assignee: Konica Minolta, Inc., Tokyo (JP)

(21) Appl. No.: 15/866,550

(22) Filed: Jan. 10, 2018

(30) Foreign Application Priority Data
Jan. 10, 2017 (JP) 2017-002044

(51) Int. Cl. G01S 7/52; A61B 8/00; A61B 8/08

(52) U.S. Cl. CPC G01S 7/52079; A61B 8/5207; A61B 8/4272; A61B 8/4444

(57) ABSTRACT

An ultrasound probe to be connected to an ultrasound diagnostic apparatus body, including an acoustic element that mutually converts an electrical signal and an ultrasonic signal, a motor that causes the acoustic element to perform rocking rotation, a frame that is formed from a resin with thermal conductive properties and that holds the acoustic element so as to allow rocking thereof, and an acoustic window that is formed from an ultrasound-transmitting material and that forms, together with the frame, a closed space that contains the acoustic element and a coupling liquid such that the motor is disposed outside the closed space.

### CROSS REFERENCE TO RELATED APPLICATIONS

Japanese Patent Application No. 2017-002044 filed on Jan. 10, 2017, including description, claims, drawings, and abstract the entire disclosure is incorporated herein by reference in its entirety.

### TECHNOLOGICAL FIELD

The present invention relates to an ultrasound probe of an ultrasound diagnostic apparatus utilizing ultrasound, and an ultrasound diagnostic apparatus.

### BACKGROUND

Ultrasound diagnostic apparatuses that irradiate the inside of test objects with ultrasound, and receive and analyze the reflected waves to inspect the inside of the test objects have been widely used. Such ultrasound diagnostic apparatuses can examine test objects non-destructively and non-invasively, and thus are widely employed in various applications, such as a medical-purpose inspection and an inspection of the inside of architectural constructions.

In an ultrasound diagnostic apparatus, a plurality of acoustic elements (transducers) that convert voltage signals into ultrasonic vibration and vice versa are arranged in a predetermined direction (scanning direction), and the acoustic elements emit ultrasound upon application of a driving voltage. Such an ultrasound diagnostic apparatus can obtain two-dimensional data in nearly real-time by switching (scanning), over time, acoustic elements that detect voltage changes due to incidence of reflected ultrasound.

Further, there exists a technique for obtaining three-dimensional images in nearly real-time by moving arranged acoustic elements back and forth (rocking) perpendicularly to the scanning direction on the emission/incident surface of ultrasound. By obtaining three-dimensional images using such a technique, operators can more easily know three-dimensional shapes and/or positional relationships of test objects, which are difficult to perceive in two-dimensional images. Ultrasound probes used in such a technique are disclosed in Japanese Patent Application Laid-Open No. 2010-131068, Japanese Patent Application Laid-Open No. 2007-289315, Japanese Patent Application Laid-Open No. 2004-016750, and Japanese Patent Application Laid-Open No. 2001-104356, for example.

### SUMMARY

An ultrasound probe includes, in part of a case thereof, an ultrasound emission surface (also referred to as acoustic window, for example) through which ultrasound generated by an acoustic element is emitted outside the ultrasound probe. By bringing the ultrasound emission surface into contact with a test object, the inside of the test object is irradiated with ultrasound. An ultrasound coupling liquid for preventing attenuation of ultrasound is filled between the acoustic element and the ultrasound emission surface.

The acoustic element generates ultrasound by using applied driving voltage. The acoustic element, however, cannot convert all the applied driving voltage into ultrasound, and part of the driving voltage is emitted as heat from the acoustic element to the surroundings. In some cases, such heat raises the temperature on the ultrasound emission surface through the ultrasound coupling liquid. Since the ultrasound emission surface is a portion that comes into direct contact with a test object, suppressed temperature rise on the ultrasound emission surface is needed.

In one or more embodiments of the present invention, an ultrasound probe that suppresses a temperature rise on an ultrasound emission surface, and an ultrasound diagnostic apparatus.

In one or more embodiments, an ultrasound probe to be connected to an ultrasound diagnostic apparatus body, includes: an acoustic element that mutually converts an electrical signal and an ultrasonic signal; a motor that causes the acoustic element to perform rocking rotation; a frame that is formed from a resin with thermal conductive properties and that holds the acoustic element so as to allow rocking thereof; and an acoustic window that is formed from an ultrasound-transmitting material and that forms, together with the frame, a closed space that contains the acoustic element and a coupling liquid such that the motor is disposed outside the closed space.

An ultrasound diagnostic apparatus reflecting one or more embodiments of the present invention includes the ultrasound probe and the ultrasound diagnostic apparatus body, in which the ultrasound diagnostic apparatus body causes the ultrasound probe to transmit an ultrasonic transmission signal to a test object, and generates an ultrasonic image on the basis of an ultrasonic reception signal generated by the ultrasound probe that has received a reflected wave from the test object.

### BRIEF DESCRIPTION OF THE DRAWINGS

The features provided by one or more embodiments of the invention will become more fully understood from the detailed description given hereinbelow and the appended drawings which are given by way of illustration only, and thus are not intended as a definition of the limits of the present invention:

FIG. 1 illustrates a configuration of an ultrasound diagnostic apparatus according to one or more embodiments of the present invention;

FIG. 2 illustrates a configuration of an ultrasound probe unit according to one or more embodiments of the present invention;

FIG. 3 is a partially-sectional perspective view illustrating a structure of the ultrasound probe according to one or more embodiments;

FIG. 4A is an exploded perspective view illustrating shapes and positional relationships of a frame, a motor, and a base according to one or more embodiments;

FIG. 4B is an exploded perspective view illustrating the shapes and the positional relationships of the frame, the motor, and the base according to one or more embodiments;

FIG. 5 is an exploded perspective view illustrating a configuration from the frame to an opening of the ultrasound probe according to one or more embodiments;

FIG. 6A is a graph that compares temperature rises on the surface of an acoustic window during operation of the ultrasound probe when different frame materials are used for the ultrasound probe according to one or more embodiments; and

FIG. 6B is a graph that compares temperature rises on the surface of the acoustic window during operation of the ultrasound probe when highly thermally conductive PPS employed as a frame material of the ultrasound probe has different thermal conductivities according to one or more embodiments.

### DETAILED DESCRIPTION OF EMBODIMENTS

Hereinafter, one or more embodiments of the present invention will be described with reference to the drawings. However, the scope of the invention is not limited to the disclosed embodiments.

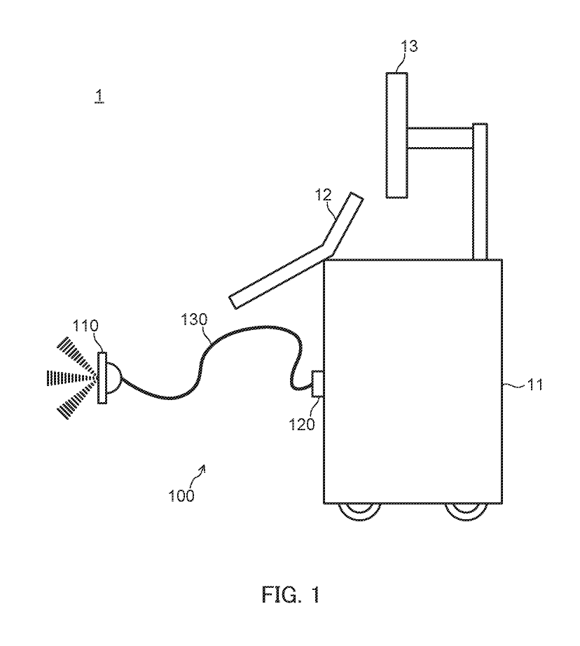

FIG. 1 illustrates a configuration of an ultrasound diagnostic apparatus according to one or more embodiments of the present invention. As illustrated in FIG. 1, ultrasound diagnostic apparatus 1 includes ultrasound probe unit 100, ultrasound diagnostic apparatus body 11, operation section 12, and display section 13. Ultrasound probe unit 100 includes ultrasound probe 110, connector 120, and cable 130.

Ultrasound probe 110 transmits ultrasound (transmission ultrasound) inside a test object (not shown), such as a living body, and receives a reflected wave, i.e., ultrasound reflected inside the test object (reflected ultrasound: echo).

Ultrasound diagnostic apparatus body 11, which is connected to ultrasound probe 110 via cable 130 and connector 120, transmits an electrical driving signal to ultrasound probe 110 to cause ultrasound probe 110 to transmit an ultrasound transmission signal to a test object, and generates an ultrasonic image of the internal state of the test object on the basis of an ultrasound reception signal generated by ultrasound probe 110 that has received a reflected wave from the inside of the test object.

Operation section 12 is an operation device, such as a switch, a button, a keyboard, a mouse, or a touch panel, and receives operations by users of ultrasound diagnostic apparatus 1, such as doctors and technicians.

Display section 13 is a display device, such as a liquid crystal display (LCD) or an organic EL display, and shows ultrasonic images generated by ultrasound diagnostic apparatus body 11 and/or various screens corresponding to the status of ultrasound diagnostic apparatus 1. An ultrasound diagnosis is made, for example, by doctors who see ultrasonic images shown in display section 13.

<Configuration of Ultrasound Probe Unit 100>

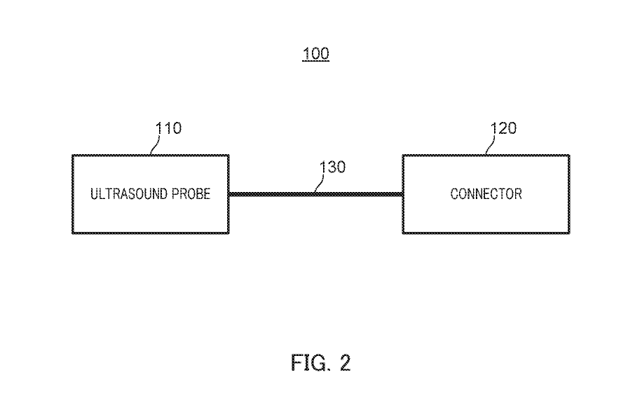

FIG. 2 illustrates a configuration of ultrasound probe unit 100 according to one or more embodiments of the present invention. As illustrated in FIG. 2, ultrasound probe unit 100 includes ultrasound probe 110, connector 120, and cable 130. Ultrasound probe unit 100 is connected to the ultrasound diagnostic apparatus body (not shown) via connector 120.

Ultrasound probe 110 comes into contact with a test object during ultrasound diagnosis, transmits transmission ultrasound, receives reflected ultrasound, and generates an ultrasound reception signal. Transmission ultrasound is generated, for example, on the basis of control signals transmitted from ultrasound diagnostic apparatus body 11 via connector 120 and cable 130. Meanwhile, ultrasound reception signals generated by ultrasound probe 110 are transmitted to ultrasound diagnostic apparatus body 11 via cable 130 and connector 120. Through the above process, ultrasonic images are generated in ultrasound diagnostic apparatus body 11.

<Description of Ultrasound Probe 110>

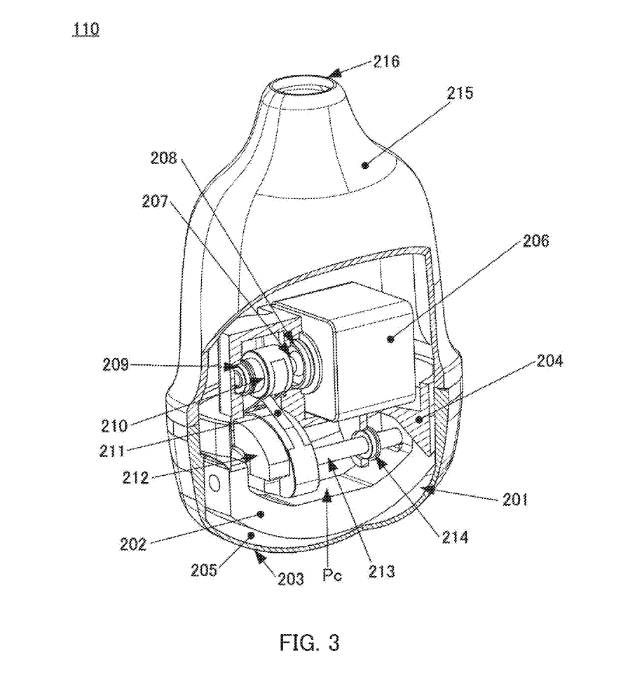

FIG. 3 is a partially-sectional perspective view illustrating a structure of ultrasound probe 110. As illustrated in FIG. 3, ultrasound probe 110 includes acoustic element 201, base 202, acoustic window 203, frame 204, coupling liquid 205, motor 206, output shaft 207, oil seal 208, bearing 209, pulley 210, belt 211, pulley 212, shaft 213, bearing 214, housing 215, and opening 216.

Acoustic element 201 is an acoustic element array in which a plurality of acoustic elements (transducers), which convert electrical signals to ultrasound and vice versa, are arranged linearly in the scanning direction. Acoustic element 201 is arranged along an arc of base 202 that is formed as a nearly circular arc. Both ends of the circular arc of base 202 are fixed to frame 204 by shaft 213 described hereinafter and rotatably held by bearing 214. The side surface portion of base 202 is formed to become flat (the reason will be described hereinafter).

Acoustic window 203 is a portion provided on the leading end of ultrasound probe 110 and is directly pressed against a test object so as to avoid attenuation of ultrasound by the air during use of ultrasound diagnostic apparatus 1. Acoustic window 203 is formed from a material that propagates transmission ultrasound generated by acoustic element 201 and reflected ultrasound reflected by a test object without attenuation. Although the details will be described hereinafter, acoustic window 203 is also formed from a material that is less likely to conduct heat (have low thermal conductivity).

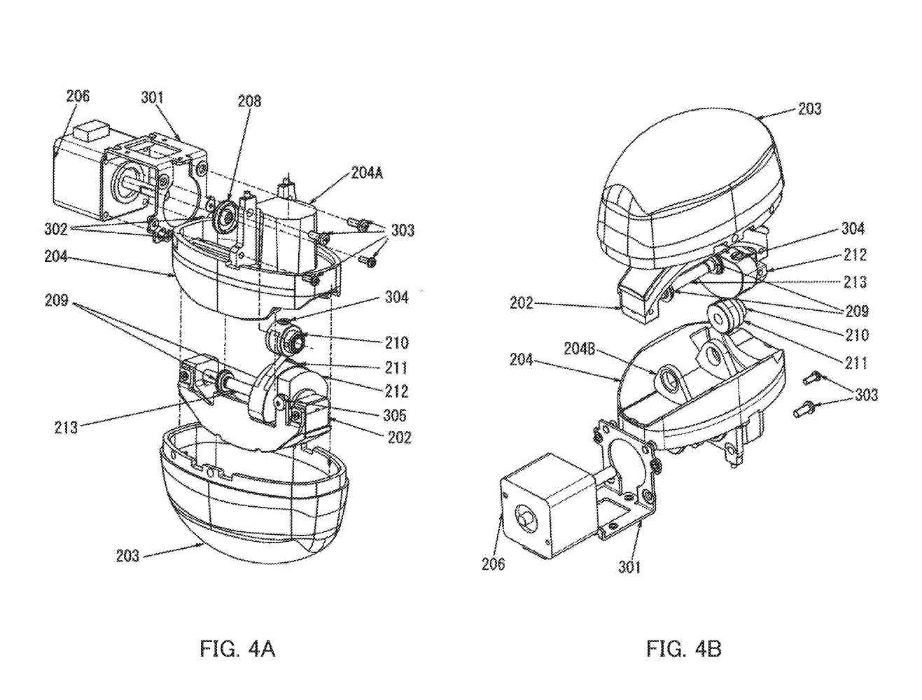

Frame 204 is a component for holding each component of ultrasound probe 110. In FIG. 3, frame 204 is partially sectioned so as to illustrate other components. The shape of frame 24 and its positional relationship with other components will be described in connection with FIGS. 4A and 4B described hereinafter.

Frame 204 is formed from a material that readily conducts heat (have high thermal conductivity) such that heat generated during operation of acoustic element 201 (ultrasound conversion) is conducted to the side of housing 215, but not to the side of acoustic window 203. Heat release by frame 204 will be described in detail hereinafter.

Closed space Pc is formed inside ultrasound probe 110 by frame 204 and acoustic window 203. Inside closed space Pc, the above-mentioned acoustic element 201 and base 202 are disposed. Moreover, the inside of closed space Pc is filled with coupling liquid 205. The purpose of coupling liquid 205 is to suppress attenuation of ultrasound.

Motor 206 is a power source for rocking base 202, on which acoustic element 201 is arranged, in a direction perpendicular to the scanning direction of acoustic element 201. Motor 206 is a stepping motor, for example, and operates on the basis of a control signal from ultrasound diagnostic apparatus body 11. Motor 206 is provided inside housing 215 of ultrasound probe 110 and outside closed space Pc. Output shaft 207 of motor 206 is inserted into closed space Pc via oil seal 208.

The tip of output shaft 207 is connected to pulley 210. The tip of output shaft 207 is rotatably held by frame 204 via bearing 209. Pulley 210 and pulley 212 are linked through belt 211, and pulley 212 is fixed to base 202. Shaft 213 penetrates both ends of base 202 and is rotatably connected to frame 204 via bearing 214. Shaft 213 is a component that functions as a central axis of the rotational motion of base 202. Output shaft 207, bearing 209, pulley 210, belt 211, pulley 212, shaft 213, and bearing 214 are provided inside closed space Pc.

Housing 215 is an exterior section of ultrasound probe 110. As described above, acoustic window 203 is provided on the leading end of ultrasound probe 100, which is part of housing 215. Acoustic window 203 and housing 215 excluding acoustic window 203 are formed from different materials. Specifically, housing 215 is formed from a material with higher thermal conductivity than a material for acoustic window 203.

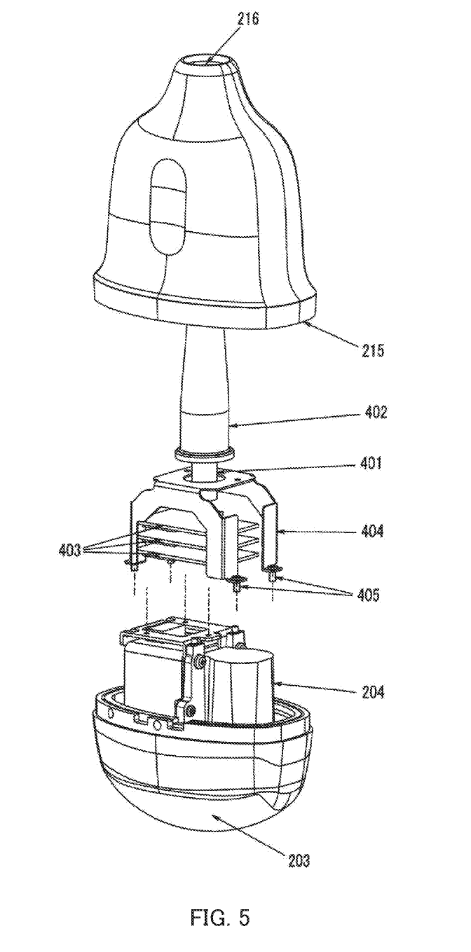

Opening 216 is provided on the base end of housing 215 for passing through bush 402 (see FIG. 5) described hereinafter.

By the above configuration, the rotational torque output by motor 206 is transmitted to shaft 213 via output shaft 207 of motor 206, pulley 210, belt 211, and pulley 212. Accordingly, base 202 makes an oscillating rotary motion around shaft 213 as a central axis. Such operation rocks acoustic element 201 and moves an ultrasound-forming surface composed of acoustic element 201, which is an acoustic element array, thereby realizing three-dimensional scanning.

<Shape and Positional Relationship of Frame 204>

Next, the shape of frame 204 and its positional relationship with other components of ultrasound probe 110 will be described in further detail.

FIGS. 4A and 4B are exploded perspective views illustrating the shapes and the positional relationships of frame 204, motor 206, and base 202. FIG. 4A illustrates each component such that acoustic window 203 is located on the lower side of the figure, whereas FIG. 4B illustrates each component such that acoustic window 203 is located on the upper side of the figure.

As illustrated in FIG. 4A, motor 206 is fixed to motor bracket 301 by motor fixing screws 302, and motor bracket 301 is fixed to frame 204 by motor bracket fixing screws 303. As illustrated in FIG. 4A, frame 204 includes protruded portion 204A protruded on the side of motor 206, and motor bracket 301 is fixed to screw holes provided on protruded portion 204A. Output shaft 207 of motor 206 is inserted into protruded portion 204A via oil seal 208. Protruded portion 204A is formed so as to have a cavity inside, and the above-mentioned pulley 210, a rotating shaft (not shown) of pulley 210, and belt 211, for example, are arranged inside protruded portion 204A. Closed space Pc is formed by frame 204 of the above-mentioned shape and acoustic window 203. In other words, closed space Pc is a space on the lower side of frame 204 inside ultrasound probe 110 in FIG. 4A, and a space on the upper side of frame 204 inside ultrasound probe 110 in FIG. 4B.

Belt 211 is fixed to pulley 210 and pulley 212 by belt fixing screw 304. Further, pulley 212 linked with pulley 210 through belt 211 is fixed to base 202 by pully fixing screw 305.

As illustrated in FIG. 4B, connecting component 204B for rotatably connecting shaft 213 is formed on frame 204 on the side of closed space Pc.

<Other Components of Ultrasound Probe 110>

FIG. 5 is an exploded perspective view illustrating a configuration from frame 204 to opening 216 of ultrasound probe 110.

Bush 402 passes through opening 216. Bush 402 is a component for protecting cable 401. Cable 401 is the same as cable 130 illustrated in FIG. 2, and is a communication line for communications, for example, between ultrasound probe 110 and ultrasound diagnostic apparatus body 11.

The end of cable 401 on the side of ultrasound probe 110 is connected to relay boards 403. Control circuits that can communicate with ultrasound diagnostic apparatus body 11 via cable 401 (130) are provided on relay boards 403. Either end of a flexible printed circuit (FPC) is connected to relay board 403 (FPC is not shown). The other end of FPC is inserted into closed space Pc through an opening (not shown) provided on protruded portion 204A of frame 204, and is connected to acoustic element 201. The opening is sealed (not shown) so as to prevent leakage of coupling liquid 205 from closed space Pc.

Relay boards 403 are held by chassis 404. Chassis 404 is fixed to motor bracket 301 by chassis fixing screws 405.

The above configuration enables, for example, transmission of a control signal from ultrasound diagnostic apparatus body 11 to acoustic element 201 inside closed space Pc, transmission of an ultrasound reception signal generated by acoustic element 201 to ultrasound diagnostic apparatus body 11, and control of operations of motor 206 on the basis of a control signal from ultrasound diagnostic apparatus body 11.

<Heat Release by Frame 204>

In the foregoing, the configuration of ultrasound probe 110 according to one or more embodiments of the present disclosure is described. In the following, suitable heat release that can be achieved by frame 204 in ultrasound probe 110 according to one or more embodiments of the present disclosure will be described in detail.

In ultrasound probe 110, when acoustic element 201 converts electrical signals into ultrasound, all the electrical signals cannot be converted to ultrasound and part of the electrical signals are rereleased as heat. Further, when motor 206 performs a rotational motion by supplied electric power, part of the electric power is released as heat. In ultrasound probe 110 according to one or more embodiments of the present disclosure, frame 204 can suitably release heat generated by these two components.

As already described in connection with FIGS. 3, 4A, and 4B, closed space Pc is formed by frame 204 and acoustic window 203, and the inside of closed space Pc is filled with coupling liquid 205.

What are in direct contact with acoustic element 201 are base 202 and coupling liquid 205. Base 202 is also in contact with coupling liquid 205. When acoustic element 201 generates heat, part of coupling liquid 205 in contact with acoustic element 201 is heated with heat from acoustic element 201.

During operation of ultrasound probe 110, base 202, on which acoustic element 201 is arranged, is rocked (oscillating rotary motion) by motor 206. By such an oscillating rotary motion of base 202, coupling liquid 205 is stirred. As described above, since the side surface portion of base 202 is formed as a flat shape, coupling liquid 205 can be stirred efficiently. This stirring continually keeps replacing coupling liquid 205 that comes into contact with acoustic element 201 by cooler coupling liquid 205. Accordingly, acoustic element 201 is cooled efficiently by coupling liquid 205.

Coupling liquid 205 heated by acoustic element 201 moves inside closed space Pc by stirring, and thus comes into contact with acoustic window 203 and frame 204. As described above, however, since acoustic window 203 is formed from a material with relatively low thermal conductivity, the amount of heat conduction from coupling liquid 205 to acoustic window 203 is relatively small. Accordingly, a temperature rise of acoustic window 203 is suppressed.

Since frame 204 is formed from a material with relatively high thermal conductivity, heat of coupling liquid 205 is efficiently conducted to frame 204 when heated coupling liquid 205 comes into contact with frame 204. Consequently, coupling liquid 205 is efficiently cooled by frame 204.

Since coupling liquid 205 is stirred by rocking of acoustic element 201 (base 202), coupling liquid 205 that comes into contact with frame 204 is continually replaced by warmer coupling liquid 205. Accordingly, warmer coupling liquid 205 heated by acoustic element 201 always comes into contact with frame 204. Consequently, heat of coupling liquid 205 is conducted to frame 204 further efficiently.

As described above, the majority of heat generated by acoustic element 201 is efficiently conducted to frame 204 without being radiated from acoustic window 203. Consequently, a rise in the surface temperature of acoustic window 203 and thus uncomfortable feelings experienced by a subject who comes into direct contact with acoustic window 203 can be avoided. Meanwhile, heat conducted to frame 204 is further conducted to housing 215 via the air inside housing 215 of ultrasound probe 110, for example. Accordingly, heat generated by acoustic element 201 is suitably radiated outside ultrasound probe 110 without remaining inside ultrasound probe 110.

As already described in connection with FIGS. 4A and 4B, frame 204 is not in direct contact with motor 206, and motor 206 is fixed to frame 204 via motor bracket 301. During operation of ultrasound probe 110, in addition to acoustic element 201, motor 206 generates heat. By forming motor bracket 301, for example, from a thermally insulating material, however, heat generated by motor 206 is less likely to be conducted to frame 204. This configuration allows the majority of heat generated by motor 206 during operation to be conducted to housing 215, for example, via the air inside housing 215 that is in contact with motor 206. Accordingly, beat that is originated from motor 206 and conducted to acoustic window 203 via frame 204 and coupling liquid 205 is reduced.

Since ultrasound probe 110 is used by being held with hands of the users of ultrasound diagnostic apparatus 1, such as doctors and technicians, it is acceptable that the surface temperature of housing 215 becomes higher than the surface temperature of acoustic window 203 that comes into direct contact with skins of subjects. Accordingly, more heat generated by motor 206 is conducted to housing 215 than to frame 204. Specifically, an upper limit of the surface temperature of housing 215 is 48° C., and an upper limit of the surface temperature of acoustic window 203 is 43° C., for example.

<Materials for Frame 204>

As described above, frame 204 is formed from a material with higher thermal conductivity than materials for acoustic window 203, for example. In the following, materials for frame 204 will be described in detail.

Examples of materials with relatively high thermal conductivity include metals. Since ultrasound probe 110 is used by being held with hands of users, such as doctors and technicians, there is a need for lighter-weight ones. Examples of light-weight metals include aluminum. When frame 204 having a complicated shape as illustrated in FIGS. 4A and 4B is manufactured by cutting out aluminum, for example, the manufacturing cost of frame 204 becomes extremely high.

Examples of materials manufactured in relatively low costs include resins. Examples of resins with excellent mechanical strength, rigidity, fire resistance, chemical resistance, electric properties, and dimensional stability include polyphenylene sulfide (PPS) resin. PPS resin, however, has relatively low thermal conductivity (0.1 to 0.5 W/m·K, for example). Accordingly, the use of PPS resin as a material for frame 204 would be undesirable since heat generated by acoustic element 201 is less likely to be conducted to frame 204 through coupling liquid 205.

In view of the above, ultrasound probe 110 according to one or more embodiments of the present invention employs PPS resin containing carbon, such as carbon fibers, carbon nanotubes, or graphite: and/or a mineral, such as copper, silver, silicon, silicon carbide, aluminum nitride, boron nitride, silicon nitride, magnesium oxide, alumina, or a low-melting alloy (Ag—Sn, Ca—Li, Al—Li, Bi—Sn, Sn—Bi—Cu—Ni, Sn—Ag—Cu—Ni, Sn—Ag—Bi—In, Mn—Sn, Mg—Sn, Mg—Zn, Al—Sn, Cu—Sn), as a relatively inexpensive material with relatively high thermal conductivity. By adding carbon and/or a mineral to PPS resin and suitably dispersing such an additive in PPS resin, PPS resin with relatively high thermal conductivity (highly thermally conductive PPS) can be prepared.

In one or more embodiments of the present invention, frame 204 is formed from PPS resin having a thermal conductivity of 5-30 W/m·K. In one or more embodiments of the present invention, a type and/or an amount of an additive (carbon, a mineral) added to PPS resin as a material for frame 204 is not particularly limited as long as the above conditions are satisfied. In other words, only carbon or only a mineral may be added to PPS resin, or both carbon and a mineral may be added to PPS resin. Further, PPS resin containing other additives excluding the above-mentioned examples may be used as a material for frame 204. In one or more embodiments of the present invention, a manufacturing method of highly thermally conductive PPS is also not particularly limited.

By forming frame 204 from a material with relatively high thermal conductivity, heat generated by acoustic element 201 is readily conducted to frame 204 via coupling liquid 205, and the temperature of coupling liquid 205 is readily lowered. Accordingly, the surface temperature of acoustic window 203 can be prevented from becoming excessively high. As a reference, acoustic window 203 of ultrasound probe 110 according to one or more embodiments of the present invention is formed from a material, such as a resin having a thermal conductivity of 0.5 W/m·K or lower (polymethylpentene, for example). In other words, the thermal conductivity of acoustic window 203 is extremely low compared with the thermal conductivity of frame 204.

Further, by employing highly thermally conductive PPS as a material for frame 204, not only the thermal conductivity of frame 204 can be raised, but also the emissivity of frame 204 can be raised moderately. When the above-described highly thermally conductive PPS is employed as a material for frame 204, for example, the emissivity is 80 to 90%.

Since the thermal conductivity and the emissivity of frame 204 are relatively high, frame 204 readily absorbs heat generated by acoustic element 201 and readily radiates absorbed heat. Accordingly, heat generated by acoustic element 201 is readily conducted to frame 204 through coupling liquid 205, and heat conducted to frame 204 is readily conducted to housing 215 or the like through the air inside ultrasound probe 110 and/or infrared radiation, for example. Consequently, heat is suitably released from housing 215 or the like without raising the surface temperature of acoustic window 203 to a temperature higher than its upper limit by heat generated from acoustic element 201, or trapping heat inside ultrasound probe 110.

Frame 204 may be formed from a material with thermal conduction anisotropy. Specifically, frame 204 may be formed so that the thermal conductivity in a direction from the contact portion with coupling liquid 205 toward another portion is higher than thermal conductivities in other directions. Such a configuration is suitable since frame 204 readily releases heat that is generated by acoustic element 201 and that is conducted from coupling liquid 205. Further, such a configuration can impede conduction of heat generated by motor 206 to coupling liquid 205 through frame 204.

As described in the foregoing, ultrasound probe 110 according to one or more embodiments of the present invention includes: acoustic element 201 that mutually converts an electrical signal and an ultrasonic signal: motor 206 that causes acoustic element 201 to perform rocking rotation; frame 204 that is formed from a resin with high thermal conductivity and holds acoustic element 201 so as to allow rocking thereof; and acoustic window 203 that is formed from an ultrasound-transmitting material and that forms, together with frame 204, a closed space that contains acoustic element 201 and coupling liquid 205 such that motor 206 is disposed outside the closed space.

The side surface portion of base 202 is formed to become flat. A resin that forms frame 204 has higher thermal conductivity than acoustic window 203, and specifically, the thermal conductivity of a resin that forms frame 204 is 5 to 30 W/m·K. The emissivity of a resin that forms frame 204 is 80 to 90%. Further, a resin that forms frame 204 is highly thermally conductive polyphenylene sulfide (PPS) in which carbon and/or a mineral is added to PPS.

By the above configuration, ultrasound probe 110 according to one or more embodiments of the present invention can obtain the following advantages. Heat generated by acoustic element 201 during conversion of electrical signals into ultrasonic signals is conducted to part of coupling liquid 205 that is in contact with acoustic element 201. Since base 202 on which acoustic element 201 is arranged is rocked (oscillating rotary motion) by motor 206, coupling liquid 205 is efficiently stirred by the flat shape of the side surface portion of base 202. Since this stirring continually keeps replacing coupling liquid 205 that comes into contact with acoustic element 201 by cooler coupling liquid 205, acoustic element 201 is efficiently cooled by coupling liquid 205.

Coupling liquid 205 heated by acoustic element 201 moves inside closed space Pc by stirring, thereby coming into contact with acoustic window 203 and/or frame 204. A rise in temperature of acoustic window 203, however, is suppressed since acoustic window 203 is formed from a material with relatively low thermal conductivity.

Since frame 204 is formed from a material with relatively high thermal conductivity, heat of coupling liquid 205 is efficiently conducted to frame 204 when heated coupling liquid 205 comes into contact with frame 204. Accordingly, coupling liquid 205 is efficiently cooled by frame 204.

Stirring of coupling liquid 205 due to rocking of acoustic element 201 (base 202) continually keeps replacing coupling liquid 205 that comes into contact with frame 204 by warmer coupling liquid 205. Accordingly, warmer coupling liquid 205 heated by acoustic element 201 always comes into contact with frame 204. Consequently, heat of coupling liquid 205 is more efficiently conducted to frame 204.

Therefore, the majority of heat generated by acoustic element 201 is efficiently conducted to frame 204 without being radiated from acoustic window 203. Consequently, a rise in surface temperature of acoustic window 203 and thus uncomfortable feelings experienced by a subject who comes into direct contact with acoustic window 203 can be avoided.

In ultrasound probe 110 according to one or more embodiments of the present invention, motor bracket 301, which is a thermally insulating component formed from a thermally insulating material, is disposed between motor 206 and frame 204.

Such a configuration impedes conduction of heat generated by motor 206 to frame 204 and thus allows the majority of heat generated by motor 206 during operation to be conducted to housing 215 through, for example, the air inside housing 215 that is in contact with motor 206, thereby reducing heat conducted to acoustic window 203 through frame 204 and coupling liquid 205. Accordingly, a rise in surface temperature of acoustic window 203 and thus uncomfortable feelings experienced by a subject who comes into direct contact with acoustic window 203 can be avoided.

In the following, specific examples of advantages in a case in which the above-described highly thermally conductive PPS is employed as a material for frame 204 in ultrasound probe 110 according to one or more embodiments of the present invention will be described.

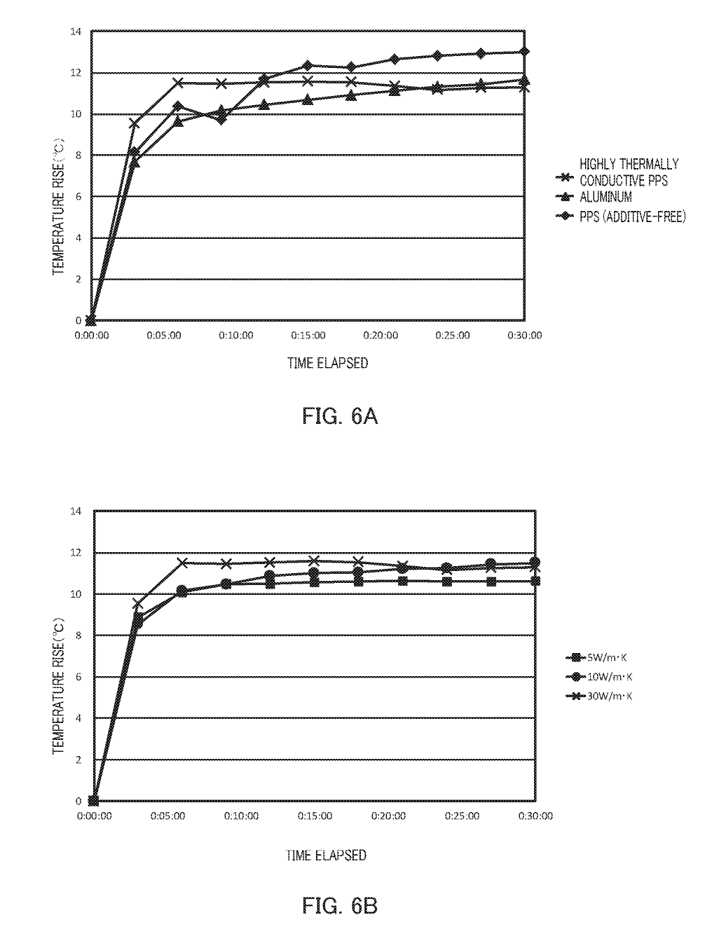

FIG. 6A is a graph that compares temperature rises on the surface of the acoustic window during operation of the ultrasound probe when different materials were used for the frame of the ultrasound probe. In FIG. 6A, “highly thermally conductive PPS” indicates a temperature change on the surface of the acoustic window in a case in which the above-described highly thermally conductive PPS was employed as a material for the frame. The highly thermally conductive PPS in FIG. 6A is PPS resin containing, as appropriate, carbon, such as graphite or carbon fibers and/or an additive, such as silver, copper, silicon, silicon carbide, aluminum nitride, boron nitride, silicon nitride, magnesium oxide, alumina, or a low-melting alloy (Sn—Bi, Sn—Bi—Cu—Ni, Sn—Ag—Cu—Ni, Sn—Ag—Cu, or Sn—Ag—Bi—In, for example). In FIG. 6A, the thermal conductivity of the highly thermally conductive PPS was 30 W/m·K. In FIG. 6A. “aluminum” indicates a temperature change on the surface of the acoustic window in a case in which aluminum was employed as a metal example of a material for the frame. In FIG. 6A, “PPS (additive-free)” indicates a temperature change on the surface of the acoustic window in a case in which additive-free PPS was employed as a material for the frame. In FIG. 6A, the temperature changes on the surface of the acoustic window were measured by an infrared thermographic camera.

As illustrated in FIG. 6A, when highly thermally conductive PPS was employed as a material for the frame, a temperature rise on the surface of acoustic window was able to be suppressed compared with the case in which additive-free PPS was employed. As also illustrated in FIG. 6A, when highly thermally conductive PPS was employed as a material for the frame, the temperature rise was able to be suppressed equally with the case in which aluminum, which is a highly thermally conductive metal, was employed.

As illustrated in FIG. 6A, the temperature on the surface of the acoustic window continues to rise as the time elapses in the case in which aluminum was employed as a material for the frame, whereas the temperature on the surface of the acoustic window becomes constant once the rise has completed in the case in which highly thermally conductive PPS was employed as a material for the frame. The reasons are as follows. When a metal that primarily releases heat through heat conduction, such as aluminum, is employed for the frame, heat of the frame is released through the air that is a low thermally conductive substance and that does not undergo convection (the air present in a narrow space around the frame). Accordingly, heat is accumulated in the air itself around the frame and is less likely to be released from the air. Consequently, heat is less likely to escape from the frame, and thus the temperature of the acoustic window continues to rise.

In contrast, in the case in which the above-described highly thermally conductive PPS was employed for the frame, since the emissivity of the highly thermally conductive PPS is relatively high as described above, exhaust heat through radiation increases as the temperature of the frame rises, and thus heat supplied to the frame and exhaust heat are balanced. Accordingly, in the case in which highly thermally conductive PPS was employed for the frame, heat is more readily released from the frame compared with the case in which aluminum (metal) was employed for the frame. Consequently, it becomes possible to prevent a temperature rise and maintain nearly a constant temperature of the acoustic window.

FIG. 6B is a graph that compares temperature rises on the surface of the acoustic window during operation of the ultrasound probe when highly thermally conductive PPS having different thermal conductivities were employed as a material for the frame of the ultrasound probe. FIG. 6B shows examples in which highly thermally conductive PPS having three different thermal conductivities of 5 W/m·K, 10 W/m·K, and 30 W/m·K were prepared by appropriately changing component(s), the amount, and/or the ratio, for example, of the above-mentioned additive(s) to PPS resin, and employed for a material for the frame.

As shown in FIG. 6B, in the case in which highly thermally conductive PPS was employed as a material for the frame, if the thermal conductivity is 5 W/m·K or higher, for example, the temperature rise on the surface of the acoustic window can be suppressed equally regardless of the magnitude of the thermal conductivity.

In the specific example shown in FIG. 6B, CZ-2060-A1 (from DIC Corporation) was used as highly thermally conductive PPS with a thermal conductivity of 5 W/m·K, H501B (from Toray Industries, Inc.) was used as highly thermally conductive PPS with a thermal conductivity of 10 W/m·K, and T121J1 (from Lion Idemitsu Composites Co., Ltd.) was used as highly thermally conductive PPS with a thermal conductivity of 30 W/m·K.

In the foregoing, one or more embodiments of the present invention are described with reference to the drawings, but the present invention is not limited to these examples. The technical scope of one or more embodiments of the present invention encompasses various variations and modifications which a person skilled in the art can conceive within the scope of the Claims. Each feature of the above-mentioned one or more embodiments may be combined specifically without departing from the spirit of the disclosure.

One or more embodiments of the present invention are suitable for an ultrasound probe of an ultrasound diagnostic apparatus that utilizes ultrasound.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. An ultrasound probe to be connected to an ultrasound diagnostic apparatus body, comprising:
an acoustic element that mutually converts an electrical signal and an ultrasonic signal;
a motor that causes the acoustic element to perform rocking rotation;
a frame that is formed from a resin with thermal conductive properties and that holds the acoustic element; and
an acoustic window that is formed from an ultrasound-transmitting material and that forms, together with the frame, a closed space that contains the acoustic element and a coupling liquid such that the motor is disposed outside the closed space.

2. The ultrasound probe according to claim 1, wherein a side surface portion of a base for the acoustic element is formed to become flat.

3. The ultrasound probe according to claim 1, wherein the resin that forms the frame has a higher thermal conductivity than the acoustic window.

4. The ultrasound probe according to claim 1, wherein the resin that Forms the frame has a thermal conductivity of 5 to 30 W/m·K.

5. The ultrasound probe according to claim 1, wherein the resin that forms the frame has an emissivity of 80 to 90%.

6. The ultrasound probe according to claim 1, wherein the resin that forms the frame is a highly thermally conductive polyphenylene sulfide (PPS) in which carbon and/or a mineral is added to PPS.

7. The ultrasound probe according to claim 1, wherein a thermally insulating component that is formed from a thermally insulating material is disposed between the motor and the frame.

8. The ultrasound probe according to claim 1, wherein the frame is formed from a material with thermal conduction anisotropy, and has a higher thermal conductivity in a direction from a contact portion with the coupling liquid toward another portion than thermal conductivities in other directions.

9. An ultrasound diagnostic apparatus comprising the ultrasound probe and the ultrasound diagnostic apparatus body according to claim 1, wherein the ultrasound diagnostic apparatus body:
causes the ultrasound probe to transmit an ultrasonic transmission signal to a test object, and
generates an ultrasonic image on the basis of an ultrasonic reception signal generated by the ultrasound probe that has received a reflected wave from the test object.
